(19) United States
(12) Patent Application Publication
(10) Pub. No.: US 2026/0335114 A1
(43) Pub. Date: Oct. 29, 2026

(54) APPARATUS FOR A WRIST BRACE THAT IS REMOVABLY ATTACHABLE TO AN ARMBOARD

(71) Applicant: Semler Technologies, Inc., Tigard, OR (US)

(72) Inventors: Philip BENZ, Leander, TX (US); Robert Henry NIEMEYER, III, Tigard, OR (US); Matthew SEMLER, Portland, OR (US); Gary MILLS, Gladstone, OR (US)

(21) Appl. No.: 19/636,898

(22) Filed: Apr. 1, 2026

Related U.S. Application Data
(60) Provisional application No. 63/781,705, filed on Apr. 1, 2025.

(51) Int. Cl. A61F 5/01

(52) U.S. Cl. CPC A61F 5/0118

(57) ABSTRACT

A wrist brace may provide for receiving a patient's hand, wrist, and forearmand be releasably attachable to an armboard. The wrist brace may include an upper surface configured for receiving the patient's wrist and forearm and a suction element coupled to the frame for releasable attachment to the armboard. The brace may include a strap coupled to the frame for securing one of the patient's hand and forearm. The suction element may include a cup with a lower surface configured for releasable suction attachment to the armboard and a semi-adhesive disposed on the lower surface of the cup, the semi-adhesive configured for releasable adhesion to the armboard. The semi-adhesive may be a circular structure centrally located on the lower surface of the cup with complementary structure on the cup and the circular structure providing a wedged or embedded connection that secures the circular structure to the cup.

### CROSS-REFERENCE TO RELATED APPLICATION

This application claims priority to U.S. Provisional Patent Application Ser. No. 63/781,705, filed Apr. 1, 2025 and entitled APPARATUS FOR A WRIST BRACE THAT IS REMOVABLY ATTACHABLE TO AN ARMBOARD, the disclosure of which is incorporated herein by reference.

### FIELD

This disclosure relates generally to wrist braces and supports and, more particularly, to novel apparatuses and methods for positioning, stabilizing, securing and extending the wrist of a human patient for the purpose of cannulating a blood vessel in the wrist and forearm.

### BACKGROUND

The following are incorporated into this application by reference: specification, claims and drawings of U.S. Pat. No. 8,652,080, “Apparatus and method of use for a wrist extension brace” by Benz et al; specification, claims and drawings of U.S. Pat. No. 10,267,350, “Integrated Armboard and Wrist Support Apparatuses and Methods of Use” by John E. Lassetter et al; product descriptions and specifications of the mBrace 4200, mBrace 4250, mBrace 4280 and mBrace 4285 at Semler Technologies, Inc.

The present disclosure is a wrist brace for use during interventional procedures involving cannulating a blood vessel in the wrist or forearm, said brace generally including a frame, a means of removably attaching to a patient's wrist that may include straps, and a means of removably attaching to an armboard that may comprise a suction element.

### BRIEF DESCRIPTION OF THE DRAWINGS

FIG. 1 (from the '080 patent)-A perspective view of the Brace 10 shows a cushion pad 30 placed on the top surface of the frame 20, included in which tie-down holes 60 and to which are attached a forearm strap 40, a hand strap 41. At the front end of the frame 20 is shown a front stabilization foot 70, and in the middle of the frame 20 is shown the narrowed waist 21.

FIG. 2 (from the '080 patent)-A perspective exploded view of the Brace 10 shows attachment means 50 and 51, for example adhesively-attached pieces of Velcro hook material, to be placed on the bottom and top surfaces of the frame 20, respectively. A front stabilization foot 70 and a rear stabilization foot 71 are also shown on frame 20 at the front and rear ends, respectively. Structural ribs 72 are also shown integrally formed into the frame 20. Also shown are the tie-down holes 60, the pad 30, the forearm strap 40, the hand strap 41 and an arm-board strap 42.

FIG. 3 (from the '080 patent)-A perspective view of the Brace 10 as deployed shows a hand 100 placed onto the Brace 10 with its dorsal surface placed flat onto the cushion pad 30. The forearm strap 40 is shown secured around the forearm 101 and the hand strap 41 is shown secured around the hand 100. Notably, the hand strap 41 wraps around the thumb proximal phalanx 102 and extends across the palm of the hand 100 and around the little finger MCP 104. Alternatively, the hand strap 41 can wrap around the thumb MCP 103 instead of the thumb proximal phalanx 102. The forearm 101, up to the wrist 105, lies over the cushion pad 30 covering the narrowed waist 21 of the frame 20.

FIG. 4a Top perspective view of a wrist brace frame. 200 wrist brace frame; 201 Distal end; 202 Openings; 203 Slots; 204 Saddle.

FIG. 4b Bottom view of a wrist brace frame. 200 wrist brace frame; 201 Distal end; 202 Openings; 203 Slots; 204 Saddle.

FIG. 4c Bottom perspective view of a wrist brace frame. 200 wrist brace frame; 201 Distal end; 202 Openings; 204 Saddle.

FIG. 4d Top view of a wrist brace frame. 200 wrist brace frame; 201 Distal end; 202 Openings; 204 Saddle.

FIG. 4e Bottom perspective view of a wrist brace frame with suction element attached. 200 wrist brace frame; 201 Distal end; 202 Openings; 204 Saddle; 250 Suction element; 251 Suction element cup.

FIG. 4f Bottom view of a wrist brace frame with suction element attached. 200 wrist brace frame; 201 Distal end; 202 Openings; 204 Saddle; 250 Suction element; 251 Suction element cup.

FIG. 4g Side view of a wrist brace frame with suction element attached. 200 wrist brace frame; 201 Distal end; 202 Openings; 204 Saddle; 250 Suction element; 251 Suction element cup.

FIG. 5a Top view of a suction element. 250 Suction element; 251 Suction element cup; 252 Suction element connector; 253 Suction element retention knob.

FIG. 5b Top perspective view of a suction element. 250 Suction element; 251 Suction element cup; 252 Suction element connector; 253 Suction element retention knob; 254 Suction element connector shaft.

FIG. 5c Bottom view of a suction element. 250 Suction element; 251 Suction element cup; 260 semi-adhesive applications on the underside of the cup 251.

FIG. 6a Top perspective view of a wrist brace frame with suction element attached. 300 wrist brace frame; 301 Distal end; 302 Openings; 303 Slots; 304 Saddle; 350 Suction element; 351 Suction element cup; 353 Suction element retention tab; 355 Suction element removal tab; 356 Suction element frame pad.

FIG. 6b Bottom view of a wrist brace frame with suction element attached. 300 wrist brace frame; 301 Distal end; 302 Openings; 303 Slots; 304 Saddle; 350 Suction element; 351 Suction element cup; 355 Suction element removal tab.

FIG. 6c Bottom perspective view of a wrist brace frame with suction element attached. 300 wrist brace frame; 301 Distal end; 302 Openings; 304 Saddle; 350 Suction element; 351 Suction element cup; 355 Suction element removal tab.

FIG. 6d Top view of a wrist brace frame with suction element attached. 300 wrist brace frame; 301 Distal end; 302 Openings; 304 Saddle; 350 Suction element; 351 Suction element cup; 353 Suction element retention tab; 355 Suction element removal tab; 356 Suction element frame pad.

FIG. 6e Bottom perspective view of a wrist brace frame. 300 wrist brace frame; 301 Distal end; 302 Openings; 304 Saddle.

FIG. 6f Bottom view of a wrist brace frame. 300 wrist brace frame; 301 Distal end; 302 Openings; 304 Saddle.

FIG. 7a Top view of a suction element. 350 Suction element; 351 Suction element cup; 352 Suction element connector; 353 Suction element retention tab; 354 Suction element shaft; 355 Suction element removal tab; 356 Suction element frame pad.

FIG. 7b Top perspective view of a suction element. 350 Suction element; 351 Suction element cup; 352 Suction element connector; 353 Suction element retention tab; 354 Suction element shaft; 355 Suction element removal tab; 356 Suction element frame pad.

FIG. 7c Side view of a suction element. 350 Suction element; 351 Suction element cup; 352 Suction element connector; 353 Suction element retention tab; 354 Suction element shaft; 355 Suction element removal tab; 356 Suction element frame pad.

FIG. 7d Side section view of a suction element attached to wrist brace frame. 300 Wrist brace frame; 301 Distal end; 302 Opening; 350 Suction element; 351 Suction element cup; 352 Suction element connector; 353 Suction element retention tab; 354 Suction element shaft; 355 Suction element removal tab; 356 Suction element frame pad.

FIG. 7e Front view of suction element with angle block. 360 Suction element; 351 Suction element cup; 352 Suction element connector; 353 Suction element retention tab; 354 Suction element shaft; 355 Suction element removal tab; 356 Suction element frame pad; 361 Angle block; 362 Angle of angle block.

FIG. 8a Front view of wrist brace frame. 300 Wrist brace frame; 301 Distal end; 302 Opening; 350 Suction element; 351 Suction element cup; 353 Suction element retention tab; 355 Suction element removal tab; 356 Suction element frame pad.

FIG. 8 b Side section view along view D of a wrist brace frame with suction element attached. 300 Wrist brace frame; 301 Distal end; 302 Opening; 350 Suction element.

FIG. 9 is a perspective view from below and the rear of a wrist brace according to another embodiment of the present disclosure, the wrist brace including a suction cup with a central ring of semi-adhesive.

FIG. 10 is an exploded version of the view of FIG. 9 showing the suction cup and semi-adhesive ring.

FIG. 11 is an exploded view from above of the embodiment of FIGS. 9 and 10.

FIG. 12 is a perspective view from below of the suction cup of FIGS. 9-11, showing the annular central groove with the annular ridge that will receive the semi-adhesive ring.

FIG. 13 is a cross-sectional view of the suction cup with the annular semi-adhesive ring in place in the annular central groove of the cup, the cup and ring being shown flipped about a horizontal axis as compared to FIGS. 9-11, and showing a central channel 485 in a connector portion of the cup that can receive a shaft of an insertion tool for coupling the cup to an opening in the frame.

FIG. 14 is a perspective view from above of the central ring of semi-adhesive.

FIG. 15 is a perspective view from above of the central ring of semi-adhesive.

FIG. 16 is a perspective view from below of a wrist brace according to another embodiment of the present disclosure, the wrist brace including a suction cup with a central ring of semi-adhesive.

FIG. 17 is a perspective view from above of a wrist brace according to another embodiment of the present disclosure, the wrist brace including a suction cup with a central ring of semi-adhesive.

FIG. 18 is a perspective view of an insertion tool 490 configured for mating with the central channel of the suction cup for coupling the cup to the frame.

### DETAILED DESCRIPTION

As described in the contents incorporated herein by reference (refer to BACKGROUND section, above), a useful wrist support brace for stabilizing, securing and extending the hand, wrist and forearm of a patient whose blood vessels in that area are about to be cannulated would have the following characteristics: i) usable for the varied usage environments including cardiac catheterization or interventional radiology laboratory, where the patient's arm, with a brace attached, would be secured to an arm-board; ii) provide stability by avoiding pronation or rolling of the forearm, wrist and hand within the brace; iii) provide stability by avoiding rolling of the brace on the arm-board or other flat surface to which it may be attached; iv) usable for critical care nursing, clinic, hospital transport bed, and surgery environment by providing compact size and shape, and comfortable contours; v) provide proper wrist extension; vi) be quick and easy to deploy; vii) be comfortable for the patient. These objectives have been achieved with the mBrace 4200, mBrace 4250, mBrace 4280 and mBrace 4285 (collectively, “mBrace”) products.

An additional objective is identified for providing increased deployment flexibility and expanded usage of wrist supports in the intended applications of assisting cannulation of blood vessels in a human patient's wrist and forearm, typically in association with an armboard to support and provide stability for the arm against movement. Options for a means of removably attaching a wrist brace to an armboard are discussed in the '350 patent, including an option for a suction cup for removable attachment. The '350 patent did not disclose any expected difficulties in implementing a suction cup attachment in a wrist brace, but in the eleven years since the filing of the application for the '350 patent, no such implementations have been forthcoming.

Difficulties that were not identified or dealt with in the '350 patent are overcome by the present disclosure. These include the need for a combination of a sufficient strength of attachment to secure the wrist brace to the armboard for the cannulization procedures typically involved with a readily detachable and repositionable mechanism, further combined with a wide flexibility in compatibility with various surfaces of different armboards. The present disclosure includes such a means attached to a wrist brace, which takes the form of a readily attachable and detachable suction element with a built-in added adhesive feature for securely coupling to the armboard without any requirement for a particular mating feature built into the armboard other than a generally flat and smooth surface.

Generally, a wrist brace 10 of the present disclosure includes a rigid or semi-rigid frame 200, 300, 400, or 500 and a suction element 250, 350, 360, 450, or 550 for removable attachment to any armboard without need for a mating feature with which to connect to on the armboard.

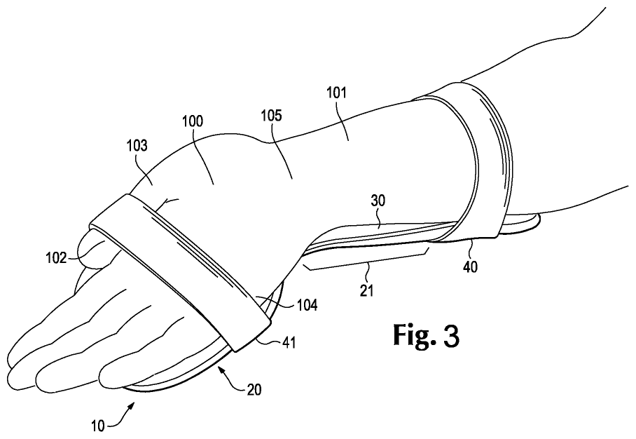

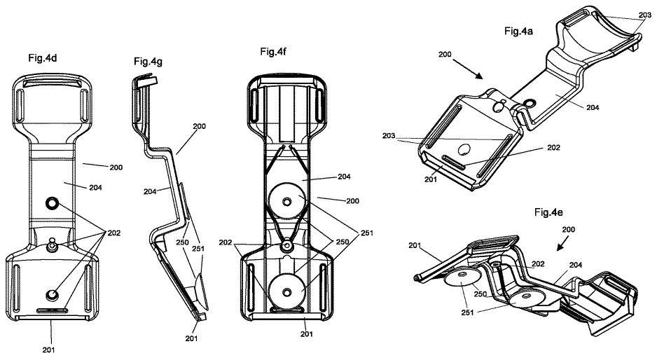

Wrist brace 10 may further include at least one of one or more straps 40 and 41 for securing it to a patient's hand and forearm, and a pad 30 placed on the top surface of the frame for cushioning. Examples of these are shown and described in the '080 patent and mBrace product descriptions incorporated herein by reference. The deployment of wrist brace 10 in FIG. 3 would similarly apply to wrist braces incorporating frames 200, 300, 400, and 500 and suction elements 250, 350, 360, 450, and 550. Preferred embodiments are not limited to those shown in the Drawings.

Frames 200, 300, and 400 may further include a saddle 204, 304, 404 for convenient attachment of a wrist hemostasis device while the wrist brace is still attached to the patient, and a clip for securing a syringe often used to adjust compression of the hemostasis device. The clip can be formed separately from the frame and then attached in a secondary operation using a press fit of a feature on the clip into a mating opening 202, 302, 402 on the frames 200, 300, and 400.

The Drawings show the frames 20, 200, 300, 400, and 500 including an extension angle that enables extension of the wrist to enhance vascular access. Alternatively, the frames 20, 200, 300, 400, and 500 may be generally flat along their longitudinal axes.

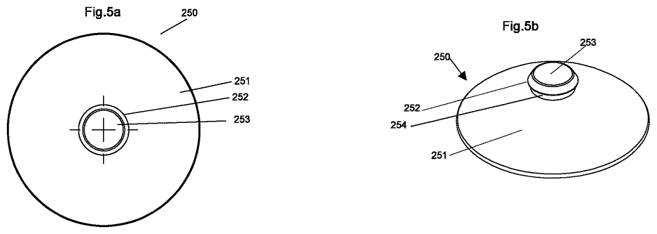

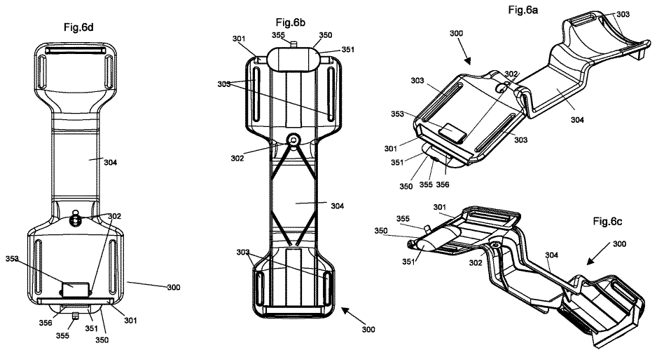

The suction elements 250, 350, 450, 550 generally include a cup 251, 351, 451, 551; a suction element connector 252, 352, 452, 552 for connection to the frames 200, 300, 400, and 500 where the cup is not integrally formed together with the brace frames 200, 300, 400, 500; and a means 355, 455, 555 of releasing the cup, e.g. a tab 355, 455, 555 for lifting up the edge thereby releasing suction. The embodiment of frame 200 may include such a tab, although it is not shown in the Drawings. A lever may further be included as part of the wrist brace assembly to assist in initiating suction by pressing the lever which an element of which presses down onto the center of the suction cups 251, 351, 451, 551—this is not shown in the Drawings. The underside of the cups 251, 351, 451, 551, i.e. the surface making contact with an armboard, may further have applied to it a semi-adhesive 260, 460, 560 to permit more aggressive attachment to the armboard while allowing removal with little or no residue. The embodiment of frame 300 may also include such a semi-adhesive although this not shown in the Drawings. The top surface of cups 251, 351, 451, 551 may further include a frame pad 356 with one or more elements as shown in FIGS. 6a-8b on which a portion of the frame rests, such as the distal ends 201 and 301 as shown for frames 200 and 300, when the suction elements 250, 350, 450, 550, separately formed, are attached at said distal ends 201 and 301.

The footprint of the cups 251, 351, 451, 551 may be any shape or size, preferred embodiments having a round, oval or obround shape and a size not exceeding five inches at their widest dimension. One or more applications of semi-adhesive 260 may be placed on the bottom surface of a suction cup 251, 351, 451, and 551 either over portions of said surface or in a ring shape or a dot or spot the width of which may be in the range of 0.1 inches to four inches. Further detail on an embodiment for the semi-adhesive is described below for frames 400 and 500.

The suction elements 250, 350, 360, 450, and 550 may be placed at any location on the underside of frames 200, 300, 400, and 500 of the wrist brace, i.e. the wrist brace surface typically facing downward towards the armboard. Various embodiments, as shown in the Drawings, describe placement under the distal ends 201, 301, 401, 501 or under their front sections. Alternative sites may be under the saddles 204, 304, 404 or under the back halves of the frames 200, 300, 400, 500. The connectors 252, 352, 452, 552 may include at least one of one or more of a shaft 254, 354, 454, 554; knob 253, 453, 553; and/or tab 353. Each suction element 250, 350, 450, 550 may have more than one connector 252, 352, 452, 552.

The suction elements 250, 350, 450, 550 or suction cups 251, 351, 451, 551 may be integrally formed onto the frames 200, 300, 400, 500, for example by overmolding, by a process known to those skilled in the art. The suction elements 250, 350, 450, 550 may also be formed separately from the frames 200, 300, 40, and 500 and then attached to the frames 200, 300, 400, 500 in a secondary operation, either during manufacturing or by a user. Such attachment may be permanent, i.e. the suction elements 250, 350, 450, 550 are not generally intended to be removable, or temporary. The means of securement may include a tab 353, knob 253, 453, 553, dart, arrowhead, barb or similar male feature on the suction elements 250, 350, 450, 550 that fits into a mating female opening on frames 200, 300, 400, 500 using a press fit. Alternatively, the male feature may be located on frames 200, 300, 400, 500 and the female element located on the suction elements 250, 350, 450, 550.

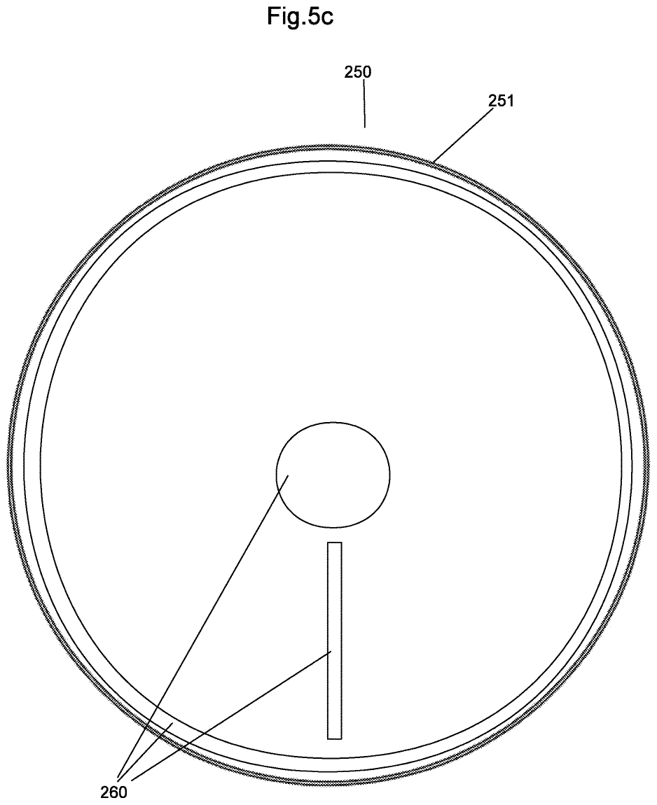

The wrist brace may further include on at least one of frames 200, 300, 400, 500 an angling block 361 wherein, during attachment to the armboard, the wrist and hand to which it is attached are positioned in one of a supine position or a semi-supine position. Alternatively, angling block 361 may form a part of the suction element, as is shown in FIG. 7e for suction element 360. The angle block 361 may present angles taken from the range of 1 degree to 90 degrees from the horizontal plane. Such positioning may alternatively be user adjustable during deployment by means of a ratchet or other adjustment mechanism in the connectors 252, 352, 452, 552 connecting frames 200, 300, 400, 500 to the suction element.

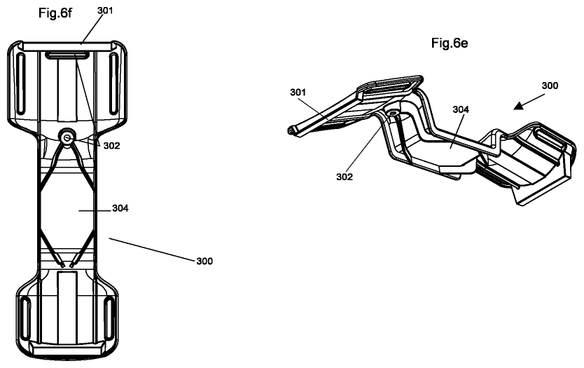

As seen in FIG. 9 and as included in the descriptions of the mBrace products incorporated herein by reference, is a syringe clip 480, which is generally permanently attached to the frame 20. Said syringe clip may likewise be included with the frames 200 or 300 by insertion into an opening 202 or 302.

The present disclosure uses a suction element 250, 350 or 360 attached to a frame 200 or 300 to achieve removable attachment of a wrist brace to the top surface of an armboard by pressing down on the top of said brace, or the top of the wrist or hand of the patient to which it is attached, to force air out from under the bottom surface of the suction cups 251 or 351, thereby achieving a suction sufficient for temporary securement to the top surface of an armboard. Lifting the tab 355 raises an edge of the cup 251 or 351 thereby permitting air to enter under the cup 251 or 351 to release the suction element 250, 350 or 360 from its removable attachment to the top surface of the armboard.

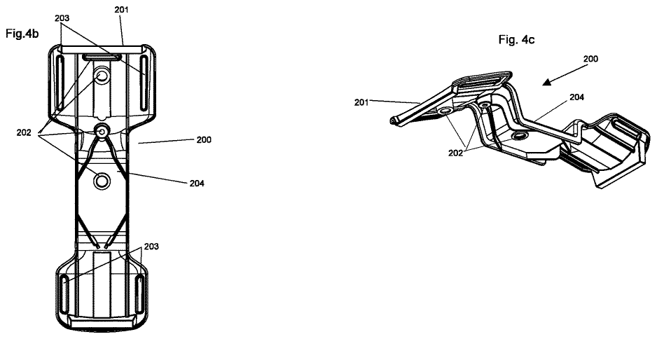

The frames 20, 200, 300, 400, and 500 may be composed of a generally rigid material, for example: a thermoplastic such as a polystyrene, polyethylene, polypropylene, nylon or polycarbonate or ABS; a fiber material; carbon fiber, 3-D printed polymer or metal or, a metal. The suction elements may be composed of an elastomer that may be formed by molding, casting, vacuum forming, or milling, examples of said elastomer including silicone, polyurethane, polyethylene, styrenic block copolymers, other thermoelastomers, rubber or the like. The side of the cups 251, 351, 451, and 551 of the suction elements 250, 350, 360, 450, and 550 that makes contact with the armboard surface may further optionally have placed on it an adhesive, more particularly, a semi-adhesive 260, 460, or 560 that permits removal of the suction elements from the armboard following deployment of the wrist brace onto said armboard with little or no remnants left on the armboard surface. There may be more than one application of this semi-adhesive 260, 460, or 560 to the underside of the suction cups 251, 351, 451, and 551, the forms including either a ring, line, stripe, spot or dot, these being placed at any location in addition to those shown in FIG. 5c. The semi-adhesive 260, 460, or 560 may also be known to those skilled in the art as a tackified silicone gel, a tacky silicone gel, a silicone tacky gel, or an isoprene polymer, styrene-butadiene copolymer, or poly(styrene-butylene-ethylene) copolymer, any of these also with added tackifiers.

One of the preferred embodiments of the present disclosure generally includes a frame 200, 300, 400, 500; a suction element 250, 350, 360, 450, 550; a saddle 204, 304, 404, 504; a syringe clip, a pad, and straps 40 and 41.

Another embodiment of the present disclosure includes an armboard attachment element that has a shape and size similar to suction elements 250, 350, 360, 450, or 550 but which does not include a suction cup 251, 351, 361, 450, 550. Instead it includes an armboard attachment pad that may have shape and size similar to said suction cups, but does not feature the suction functionality, and includes on its bottom surface a semi-adhesive 260. The armboard attachment pad may be formed separately from the frames 200, 300, 400, 500 or formed integrally with them, and may be composed of an elastomer or a rigid material similar to or the same as used in the frames 200, 300, 400, 500.

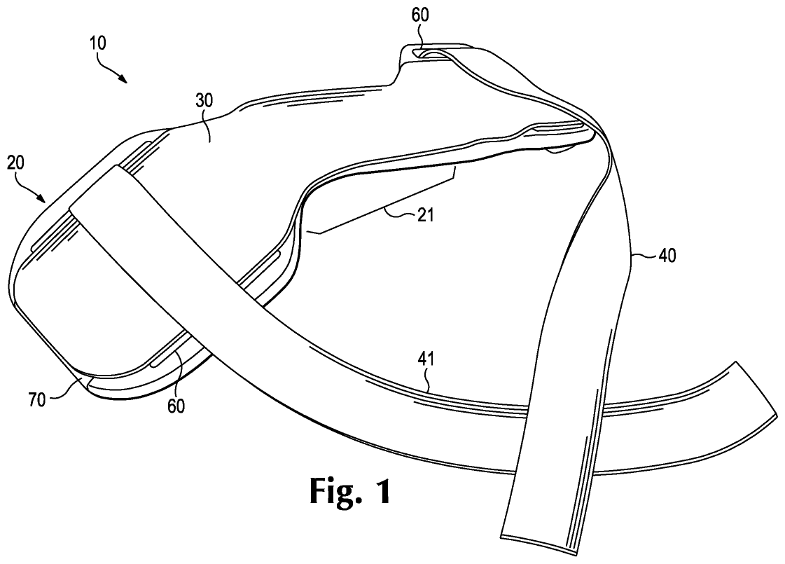

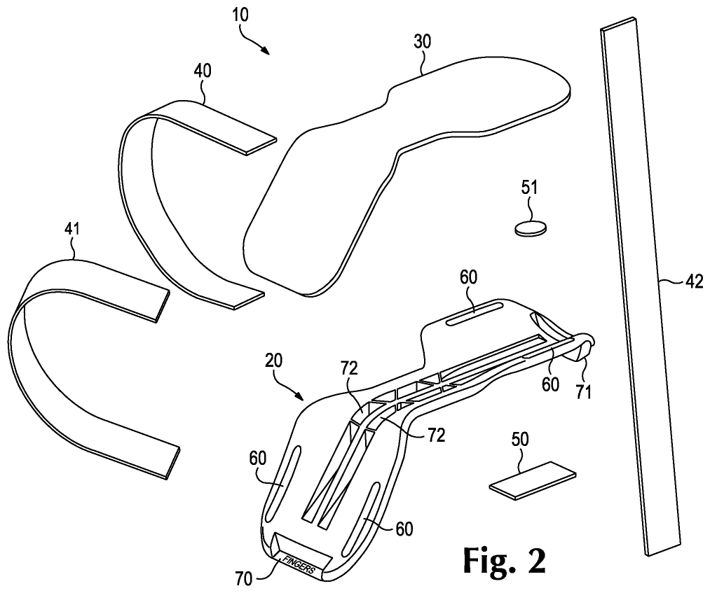

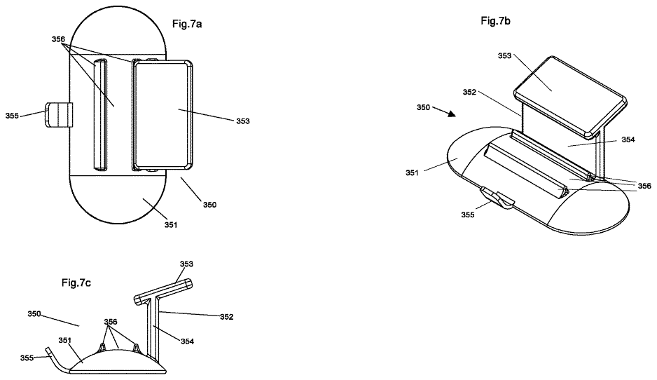

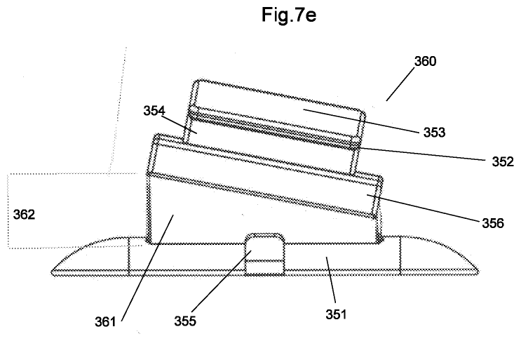

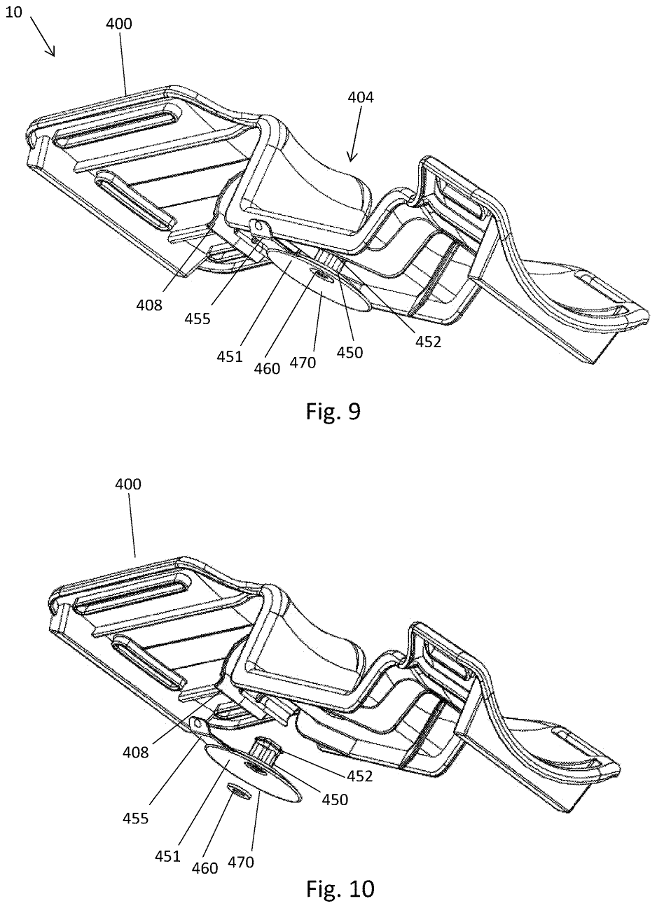

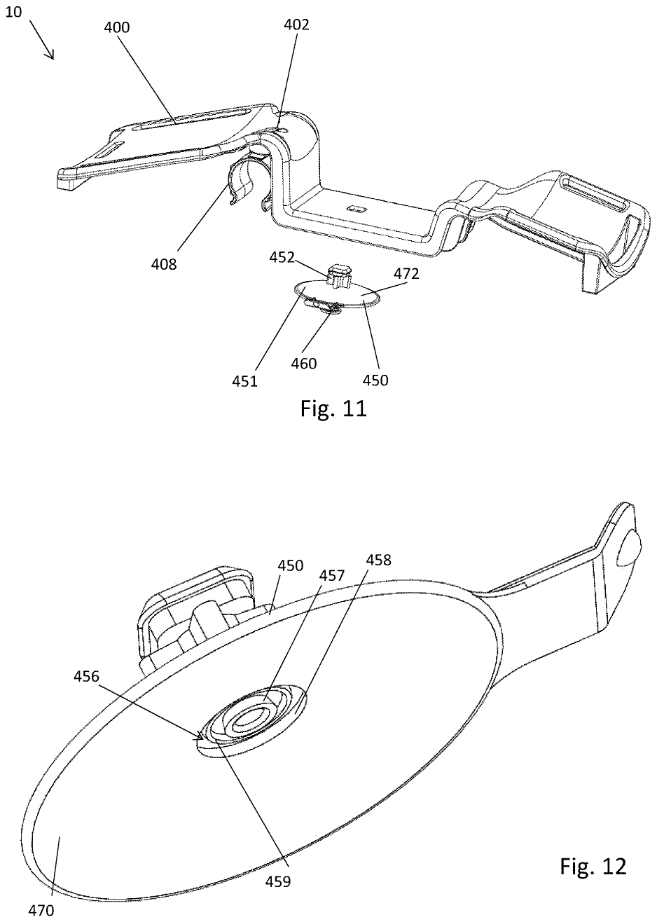

In another embodiment of the present disclosure, wrist brace 10 as shown in FIGS. 9-15 includes a rigid or semi-rigid frame 400, and a suction element 450 for removable attachment to any armboard without need for a mating feature with which to connect to on the armboard. Wrist brace 10 of this embodiment may include one or more straps 40 and 41 for securing a patient's hand and forearm, and a pad 30 placed on the top surface of the frame for cushioning, as shown in FIGS. 1-3, or in the '080 patent or mBrace product descriptions incorporated herein by reference.

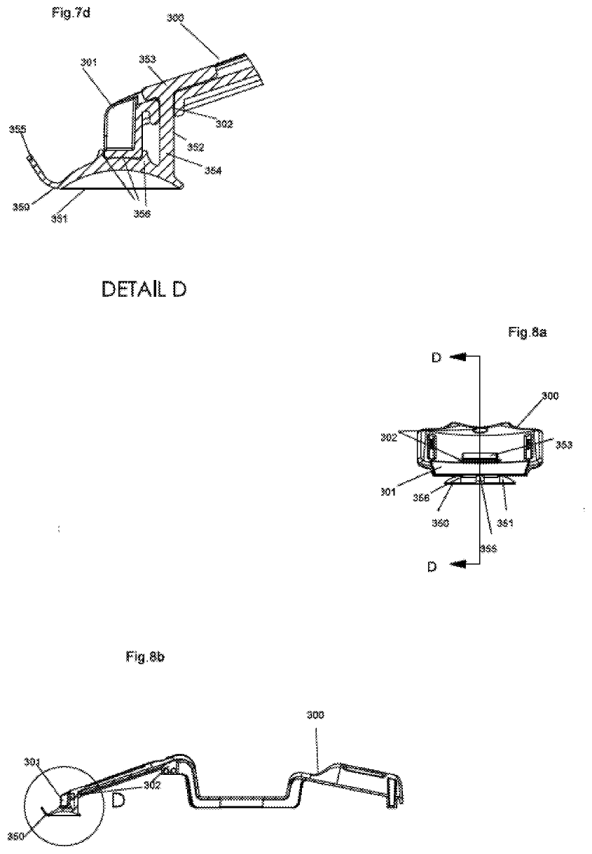

Frame 400 may further include a saddle 404 providing access space as in other embodiments for convenient attachment of a wrist hemostasis device while the wrist brace is still attached to the patient, and a clip 408 (FIG. 9-11) for securing a syringe often used to adjust compression of the hemostasis device. The clip may be formed separately from the frame and then attached in a secondary operation using a press fit of a feature on the clip into a mating opening 402 (FIG. 11) on frame 400. Frame 400 may also provide an extension angle that enables extension of the wrist to enhance vascular access. Alternatively, the frame 400 may be generally flat along its longitudinal axis.

Suction element 450 typically includes a cup 451, a suction element connector 452 for connection to frame 400 if the cup is not integrally formed together with brace frame 400, a means 455 of releasing the cup, e.g. a tab 455 for lifting up the edge thereby releasing suction. A lever (not shown) may further be included as part of the wrist brace assembly to assist in initiating suction by pressing the lever which an element of which presses down onto the center of suction cups 451.

Cup 451 may further have applied to it a semi-adhesive 460 to permit more aggressive attachment to the armboard while allowing removal with little or no residue. Semi-adhesive 460 may be applied on an underside 470 of cup 460, i.e. the surface making contact with an armboard. A top surface 472 of cup 451 may further include one or more frame pads (not shown) on which frame 400 may rest, as in the embodiment of FIGS. 6a-f, when suction element 450, separately formed, are attached to frame 400.

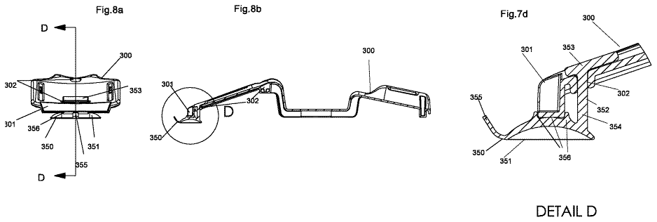

As shown in FIGS. 12-15, suction cup 451 may include an annular groove 456 defined by a pair of concentric annular frusto-conical inner surfaces 457, 458, and a ridge 459. Typically, semi-adhesive 460 is applied in a liquid form into groove 456 of cup 451. The liquid may set like an epoxy or cool to form a shape of an annular ring 461 with a first flat surface 462, a second grooved surface 463 opposite first surface 462, and a central opening 464. The annular ring of semi-adhesive may include a frusto-conical inner surface at central opening 464 and an outer frusto-conical surface 465 within groove 456. Such a structure mates the annular ring with the cup, effectively embedding the annular ring in the underside of the cup in the annular groove. The mating between the suction cup and the annular ring may be augmented by the annular ridge 459 of the cup increasing the surface area of contact with the grooved surface 463 of the ring. The cup and ring each having a frusto-conical shape complementary to the other effectively provide a connection that would be comparable to a wedged press fit if the ring were formed separately and then attached. Alternatively, the ring could be formed and mated in this manner.

The mating ridges and grooves of the suction cup and ring may alternatively be switched in location between the two parts as best suited to a particular application. The groove and opening of the semi-adhesive may provide additional surface area for adhesion between semi-adhesive 460 and suction cup 451. A bond between them may be provided by the semi-adhesive alone, by the formation of the ring within the groove or a press fit as described above, and/or may be augmented by an adhesive applied between the annular ring and the suction cup. The central location of the semi-adhesive in the suction cup may combine with the suction capabilities of the cup to provide an adherence to the armboard that is greater than the sum of the semi-adhesive adherence and the suction cup adherence provide if each were operating alone.

As noted above, the footprint of the cup 451 may be any shape or size as best suited to a particular application. As shown in FIGS. 9-13, cup 451 may be round and about 40-mm in diameter with a circular, central semi-adhesive, and other embodiments may have a round, oval or obround shape and a size. Typically, the footprint of the shapes are not exceeding five inches at their widest dimension.

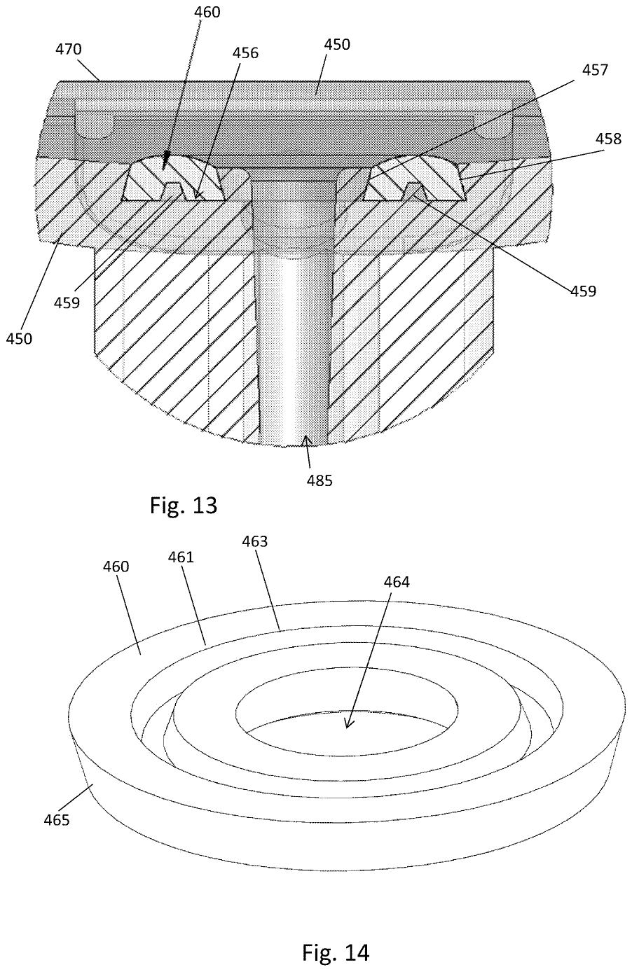

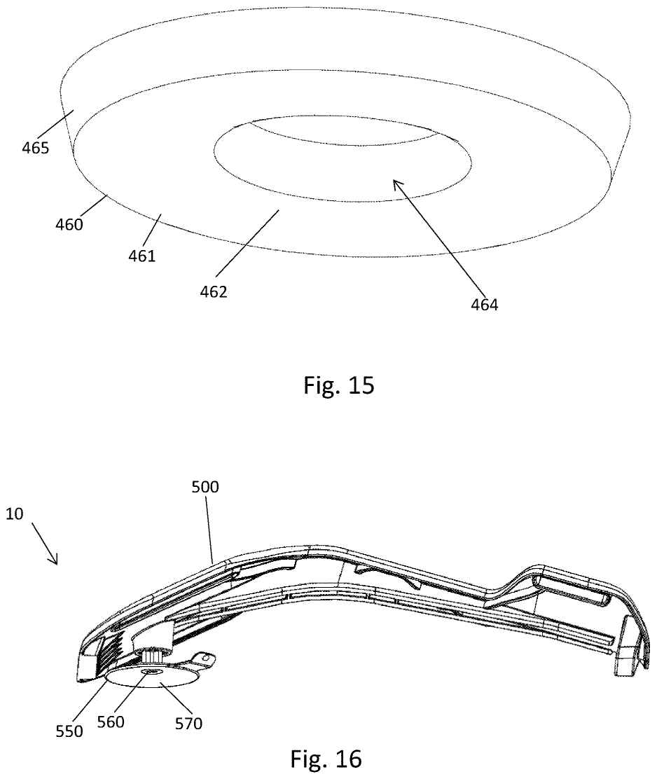

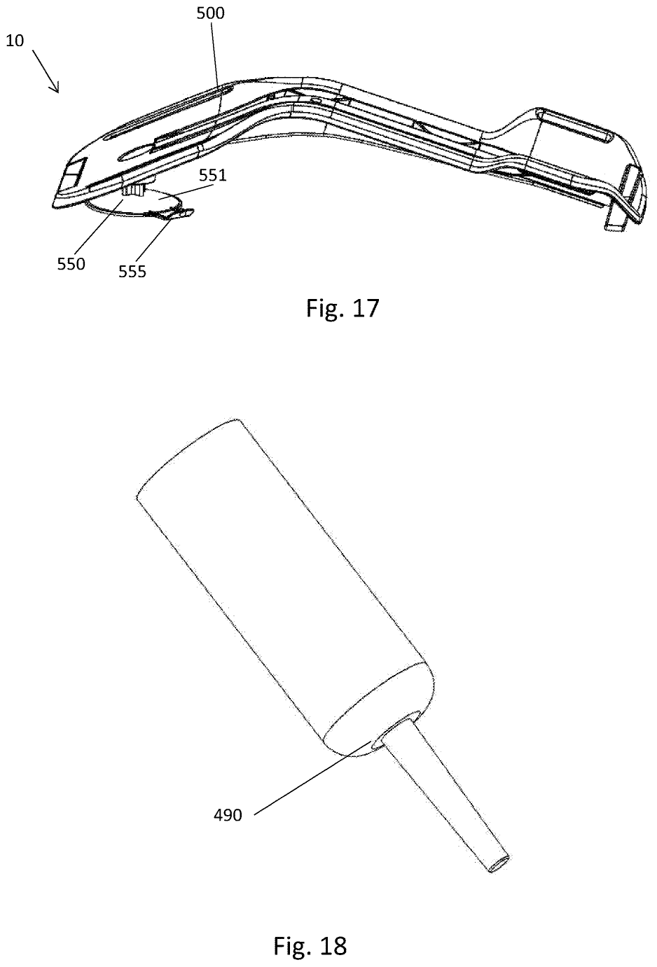

In another embodiment of the present disclosure, wrist brace 10 as shown in FIGS. 16-17 includes a rigid or semi-rigid frame 500, and a suction element 550 for removable attachment to any armboard without need for a mating feature with which to connect to on the armboard. Wrist brace 10 of this embodiment may include one or more straps 40 and 41 for securing a patient's hand and forearm, and a pad 30 placed on the top surface of the frame for cushioning, as shown in FIGS. 1-3, or in the '080 patent or mBrace product descriptions incorporated herein by reference.

Frame 500 may further include a saddle 504 providing access space as in other embodiments for convenient attachment of a wrist hemostasis device while the wrist brace is still attached to the patient, and a clip (FIG. 9) for securing a syringe often used to adjust compression of the hemostasis device. The clip may be formed separately from the frame and then attached in a secondary operation using a press fit of a feature on the clip into a mating opening 502 on frame 500. Frame 500 may also provide an extension angle that enables extension of the wrist to enhance vascular access. Alternatively, the frame 500 may be generally flat along its longitudinal axis.

Suction element 550 typically includes a cup 551, a suction element connector 552 for connection to frame 500 if the cup is not integrally formed together with brace frame 500, a means 555 of releasing the cup, e.g. a tab 555 for lifting up the edge thereby releasing suction. A lever (not shown) may further be included as part of the wrist brace assembly to assist in initiating suction by pressing the lever which an element of which presses down onto the center of suction cups 551.

Cup 551 may further have applied to it a semi-adhesive 560 to permit more aggressive attachment to the armboard while allowing removal with little or no residue. Semi-adhesive 560 may be applied on an underside 570 of cup 550, i.e. the surface making contact with an armboard. Semi-adhesive 560 may have one or more shapes for mating with one or more complementary shapes on cup 551 and features for bonding the two together and releasably attaching to an armboard, similar to that described above for semi-adhesive 460 and cup 451.

This Specification and the Drawings show embodiments of a wrist brace with suction elements 250, 350, 360, 450, 550 for the purpose of removable attachment to a patient and an armboard, but the scope of the present disclosure is not limited only to what is explicitly shown and described. It is within the scope of the present disclosure that elements shown in one embodiment may be included on another embodiment, e.g. a removal tab may be included on suction elements not specifically described above as including a removal tab, and the angle block may alternatively be included on the frames, or on the suction elements, or that two or more suction elements be included on each frame. It is further intended that the pad 30, attachment means 50 and 51, and straps 40 and 41 shown in FIGS. 1, 2 and 3, with modifications as applicable to any of the various frames, may be included in a wrist brace that includes such frames, and that the deployment onto a patient shown in FIG. 3 may function similarly with wrist braces that include any of the various frames. Likewise, features shown on, for example, the frames 200 and 300 and suction elements 250, 350, and 360, may be included with frame 20 of the brace 10, e.g. the suction elements 250, 350 and 360 may be inserted into openings 202 and 302 included on the frame 20.

Specific shapes and locations of suction cup 250, 350, 360, 450, 550; connectors 252, 352, 452, 552; knob 253 or tab 353, 453, 553; frame pad 356 may also differ somewhat from what is shown in the Drawings. Shapes and locations of openings 202, 302, 402, 502 and saddles 204, 304, 404 on frames 200, 300, 400 may differ somewhat from what is shown in the Drawings. Shapes of frames 200, 300, 400, 500 may also differ from what is shown in the Drawings.

It will be understood that the present disclosure is not limited to the method or detail of construction, fabrication, material, application or use described and illustrated herein.

It is further intended that any other embodiments of the present disclosure that result from any changes in application or method of use or operation, configuration, method of manufacture or fabrication, shape, size, or material, which are not specified within the detailed written description or illustrations contained herein yet would be understood by one skilled in the art, are within the scope of the present disclosure.

Accordingly, while the present disclosure has been shown and described with reference to the foregoing embodiments of the invented apparatus, it will be apparent to those skilled in the art that other changes in form and detail may be made therein without departing from the spirit and scope of the disclosure as defined in the appended claims.

What is claimed:

1. A wrist brace for receiving a patient's hand, wrist, and forearm, the wrist brace releasably attachable to an armboard, the wrist brace comprising:
a frame with a lower surface and having an upper surface configured for receiving a patient's wrist and forearm;
a suction element coupled to the frame, the suction element configured for releasable attachment to the armboard;
at least one strap coupled to the frame and configured for securing one of the patient's hand and forearm;
wherein the suction element includes a cup with a lower surface configured for releasable suction attachment to the armboard and a semi-adhesive disposed on the lower surface of the cup, the semi-adhesive configured for releasable adhesion to the armboard.

2. The wrist brace of claim 1 further wherein the semi-adhesive is a circular structure centrally located on the lower surface of the cup.

3. The wrist brace of claim 2 wherein the semi-adhesive is an annular ring with a first flat surface and a second surface opposite first surface, the first surface configured for facing the armboard and the second surface including a structure for coupling to the cup.

4. The wrist brace of claim 3 wherein the annular ring includes a central opening with a frusto-conical inner surface and the cup includes at least one annular surface having a frusto-conical shape complementary to that of the central opening of the annular ring.

5. The wrist brace of claim 3 wherein lower surface of the cup includes an annular groove and the annular ring is embedded in the annular groove.

6. The wrist brace of claim 3 wherein a structure on the annular ring and a complementary structure on the cup provide a wedged connection that secures the annular ring to the cup.

7. The wrist brace of claim 3 wherein the cup includes at its lower surface an annular groove defined by a pair of concentric frusto-conical surfaces and the second surface of the annular ring includes a complementary pair of frusto-conical surfaces, the frusto-conical surfaces of each combining to secure the annular ring to the cup.

8. The wrist brace of claim 3 wherein the cup includes at least one central annular ridge and the grooved surface of the annular ring mates with the at least one annular ridge of the cup.

9. The wrist brace of claim 1 wherein the cup includes a tab for lifting up the cup to release suction.

10. The wrist brace of claim 1 wherein the frame further includes a saddle providing access space for attachment of a wrist hemostasis device while the wrist brace is attached to the patient's hand and forearm.

11. The wrist brace of claim 10 further comprising a clip configured for securing a syringe for use in adjusting compression of the hemostasis device.
